(19) United States
(12) Patent Application Publication
(10) Pub. No.: US 2006/0247676 A1
(43) Pub. Date: Nov. 2, 2006

(54) Retrieval device

(75) Inventors: David Vale, Dublin (IE); Paul Gilson, Moycullen (IE); Patrick Griffin, Castlegar (IE)

Correspondence Address: SUGHRUE MION, PLLC, 2100 PENNSYLVANIA AVENUE, N.W., SUITE 800, WASHINGTON, DC, 20037, US

(73) Assignee: SALVIAC LIMITED

(21) Appl. No.: 11/475,069

(22) Filed: Jun. 27, 2006

Related U.S. Application Data
(63) Continuation of application No. 10/043,173, filed on Jan. 14, 2002.
Application No. 10/043,173 is a continuation of application No. PCT/IE99/00082, filed on Aug. 12, 1999.

(51) Int. Cl. A61M 29/00

(52) U.S. Cl. 606/200

(57) ABSTRACT

A retrieval device (1) for retrieval of an embolic filter (2) from the vasculature (3) comprises a guide catheter (42), a retrieval catheter (10) and a centring catheter (11) which is threaded over a guidewire (25). The retrieval catheter (10) comprises an elongate flexible tube (15) including a radially expansible distal tip (19) with an open mouth (40) for retrieval of the embolic filter (2). The centring catheter (11) also comprises an elongate tube (20) which extends through the retrieval catheter tube (15) and terminates in a distal tapered end (21). The centring catheter (1) is used to centre the retrieval catheter (10) in the vasculature (3) to enable the retrieval catheter (10) to freely pass through a stenosis and/or stent to retrieve the filter (2). When the retrieval catheter (10) is in position the centring catheter (11) is withdrawn through the retrieval catheter (10).

The invention relates to a retrieval device for retrieving a medical device from a body lumen. More particularly, the retrieval device may be used for retrieving an embolic filter device deployed in the vasculature of a patient to provide protection against embolic material dislodged during a surgical or medical procedure. Such an embolic protection device is described in our WO 99/23976A.

To retrieve a deployed medical device such as an embolic filter from a body lumen a retrieval catheter is used. The catheter is in the form of an elongated tube with an open mouth at the distal end into which the deployed device is retrieved. There is however a problem with such retrieval catheters in that the open mouth can become snagged at sharp changes in direction in the body lumen, at stenosed regions, or on other deployed medical devices such as stents. This is particularly the case with an embolic filter or other distal protection device which is deployed downstream of a stenosis during an angioplasty.

There is therefore a need for an improved retrieval device which will overcome this problem.

### STATEMENTS OF INVENTION

According to the invention there is provided a retrieval device for retrieving a medical device from a body lumen, through a lesion or a treatment device proximal of the medical device, the retrieval device comprising:
a retrieval catheter having a proximal end and a distal end, the retrieval catheter defining a retrieval space into which a medical device is retrieved; and
a centring means having an introduction configuration for introduction of the retrieval device through a lesion or a treatment device and a retrieval configuration in which the medical device is retrieved into the retrieval space of the retrieval catheter.

The centring means facilitates the crossing of a lesion or a treatment device such as a stent to facilitate retrieval of a medical device such as an embolic filter distal of the lesion or deployed stent.

In one embodiment of the invention the centring means is a centring catheter having a distal end, the centring catheter being mounted in the retrieval catheter for movement between the introduction configuration in which the distal end of the centring catheter projects distally from the distal end of the retrieval catheter and the retrieval configuration in which the distal end of the centring catheter is proximal of the retrieval space of the retrieval catheter.

In one embodiment of the invention the centring catheter has an internal bore extending at least partially along its length for threading the retrieval device over a guidewire. The centring catheter positions the guidewire centrally in the body lumen away from the lumen walls.

In a preferred embodiment the centring catheter is slidably movable in the retrieval catheter from the introduction configuration to the retracted retrieval configuration. This provides retrieval space in the retrieval catheter.

Ideally the centring catheter is removable from the retrieval catheter. This allows procedures such as aspiration to be carried out. Other devices, such as a snare, may also be introduced.

In another embodiment of the invention a proximal end of the centring catheter extends proximally from the proximal end of the retrieval catheter for external manipulation of the centring catheter relative to the retrieval catheter.

In a particularly preferred embodiment the distal end of the centring catheter is tapered distally inwardly to guide the open mouth through the body lumen. Ideally the distal end of the centring catheter is shaped to provide a smooth transition between the distal end of the centring catheter and the distal end of the retrieval catheter. Desirably the distal end of the centring catheter is of arrowhead shape. In this manner the retrieval device has a smooth, step free crossing profile for safe, snag-free advancement through the body lumen. Preferably the distal end of the centring catheter is flexible to facilitate navigation through the vasculature.

In another embodiment the distal end of the centring catheter is sealably engagable to the distal end of the retrieval catheter.

In an alternative aspect of the invention the retrieval catheter has a main catheter body and the centring means is a tapered distal extension of the main body of the retrieval catheter, the tapered distal extension having an open mouth through which a medical device is retrieved.

In one embodiment of this aspect of the invention at least portion of the distal extension is movable inwardly between the introduction and retrieval configurations. In this case preferably the distal extension is inverted on movement from the introduction to the retrieval configurations. Preferably the distal extension is of resilient elastomeric material.

In another embodiment of this aspect of the invention at least portion of the distal extension is movable radially outwardly between the introduction and retrieval configurations.

The distal end of the centring means may be at least partially radiopaque. This assists in guiding the distal end through the body lumen to the medical device to be retrieved.

The centring means may be of or coated with a material with a low coefficient of friction. In this way advancement of the centring means through the body lumen is eased.

In a further embodiment of the invention the retrieval catheter has a radially expansible tip at the distal end to accommodate retrieval of a medical device into the retrieval system. Thus the retrieval catheter may retrieve medical devices with a diameter greater than the relaxed diameter of the catheter. The tip may be thin-walled to ensure a low crossing profile for the retrieval device. Desirably the tip has sufficient axial stiffness to assist the retrieval of a medical device.

In another embodiment of the invention the diameter of the retrieval catheter varies along its length.

In a particularly preferred embodiment the distal end of the retrieval catheter is tapered distally inwardly to provide a smooth crossing profile for the retrieval device. This ensures that the retrieval device has a smooth, step-free crossing profile.

Preferably the medical device is mounted on or engagable with a guidewire for retrieval of the medical device into the retrieval space.

In one particularly preferred embodiment of the invention the medical device is an embolic filter device which is mounted on a guidewire for retrieval into the retrieval space.

In another preferred embodiment the inner surface of the retrieval catheter and/or the outer surface of the centring catheter is of non-circular profile over at least portion of the length thereof. Preferably, the catheter periphery is non-circular. Ideally the catheter periphery is of oval shape.

In one embodiment of the invention the catheter periphery is shaped to define a number of separate areas of contact with the other catheter. The catheter periphery may be of fluted shape.

The retrieval device of the invention is particularly suited for retrieving an embolic filter from a blood vessel.

In a further aspect the invention provides a method of a medical device from a body lumen comprising the steps of:
introducing a retrieval catheter with a centring means into a body lumen, the retrieval catheter defining a retrieval space, and the centring means having an introduction configuration for introduction of the retrieval catheter and a retrieval configuration for retrieving a medical device;
advancing the retrieval catheter across a lesion or a treatment device with the centring means in the introduction configuration;
advancing the retrieval catheter distally to the proximal end of the medical device;
retrieving the medical device into the retrieval catheter with the centring means in the retrieval configuration; and
removing the retrieval catheter and the retrieved medical device from the body lumen.

Preferably the centring means is a centring catheter and the method includes the step of moving the centring catheter from an introduction configuration in which the distal end of the centring catheter projects distally from the retrieval catheter for crossing a lesion or a treatment device and a retrieval configuration in which the centring catheter is proximal of the retrieval space for retrieving the medical device.

Preferably the retrieval catheter and the centring catheter are introduced into the body lumen over a guidewire. The centring catheter positions the guidewire centrally in the lumen away from the lumen walls.

In one embodiment of the invention the distal end of the retrieval catheter expands radially outwardly during retrieval of the medical device into the retrieval catheter. Thus the retrieval catheter may retrieve medical devices with a diameter greater than the relaxed diameter of the catheter. The distal end of the retrieval catheter may expand radially outwardly during retraction of the distal end of the centring catheter into the retrieval catheter. Typically the distal end of the centring catheter is deformed as the distal end of the centring catheter is retracted into the retrieval catheter.

In another embodiment the retrieval catheter has a main catheter body, the centring means is a tapered distal extension of the main body of the retrieval catheter and the tapered distal extension has an open mouth through which a medical device is retrieved.

In one case at least portion of the distal extension is movable inwardly between the introduction and retrieval configurations.

Alternatively at least portion of the distal extension is movable radially outwardly between the introduction and retrieval configurations.

In a preferred embodiment the method includes the step of flushing and/or aspirating before retrieving the medical device into the retrieval catheter. Ideally the method includes the step of removing the centring catheter from the retrieval catheter to facilitate flushing and/or aspiration. This provides more space within the retrieval catheter for the flushing and/or aspiration.

In one embodiment of the invention the medical device is retrieved into the retrieval catheter by drawing the medical device proximally into the retrieval catheter. In an alternative embodiment the medical device is retrieved into the retrieval catheter by advancing the retrieval catheter distally relative to the medical device. The medical device may also be retrieved by a combination of drawing the medical device proximally and advancing the catheter distally.

The centring catheter may be removed from the body lumen before removing the retrieval catheter and the medical device from the body lumen.

Ideally the centring catheter structurally supports the retrieval catheter during introduction into the body lumen. This enables a very thin-walled low-profile retrieval catheter to be used.

Preferably the medical device is mounted on or engagable with a guidewire.

In one preferred embodiment of the invention the medical device is an embolic filter device.

The method for retrieving a medical device from a body lumen according to the invention is particularly suitable for retrieving an embolic filter device from a blood vessel that has been deployed distal to an implanted stent in the treated blood vessel.

### BRIEF DESCRIPTION OF THE DRAWINGS

The invention will be more clearly understood from the following description thereof given by way of example only, in which:

FIG. 1 is a side view of a retrieval device according to the invention;

FIG. 2 is a perspective partially cut-away view of the retrieval device of FIG. 1, in use;

FIGS. 3(a) to 3(d) are side partially cut-away views of the retrieval device of FIG. 1 in different positions of use;

FIG. 4 is a diagrammatic view of the retrieval device of FIG. 1 in use;

FIG. 5 is an enlarged cross sectional view of a distal end of the retrieval device of FIG. 4;

FIG. 6 is a side cross sectional view of a distal end detail of another retrieval device according to the invention;

FIGS. 7 to 9 are schematic side views of the retrieval device of FIG. 6 in use;

FIG. 10 is a side cross sectional view of a distal end detail of another retrieval device according to the invention;

FIG. 11 is a side cross sectional view of the retrieval device of FIG. 10 in an advancement mode;

FIG. 12 is a side cross sectional view of the retrieval device of FIG. 10 in use;

FIG. 13 is a side cross sectional view of a distal end detail of yet another retrieval device according to the invention;

FIGS. 14 to 17 are schematic side views of the retrieval device of FIG. 13 in use;

FIG. 18 is a side cross sectional view of a distal end detail of a further retrieval device according to the invention;

FIG. 19 is a side cross sectional view of a distal end detail of another retrieval device according to the invention;

FIG. 20 is a perspective partially cut-away view of a detail of a catheter assembly of the invention;

FIG. 21 is a perspective partially cut-away view of a detail of another catheter assembly of the invention;

FIG. 22 is a cross sectional end view of a detail of another catheter assembly of the invention.

FIG. 23 is a side, partially cross sectional view of a retrieval device according to another embodiment of the invention;

FIGS. 24 to 26 are views of the retrieval device of FIG. 23 in use for retrieval of a distal embolic filter;

FIG. 27 is a side, partially cross sectional view of another retrieval device according to the invention;

FIGS. 28 to 30 are views of the retrieval device of FIG. 27, in use; and

FIG. 31 is a perspective view of an enlarged scale of the distal tip of the retrieval device of FIGS. 27 to 30.

### DETAILED DESCRIPTION

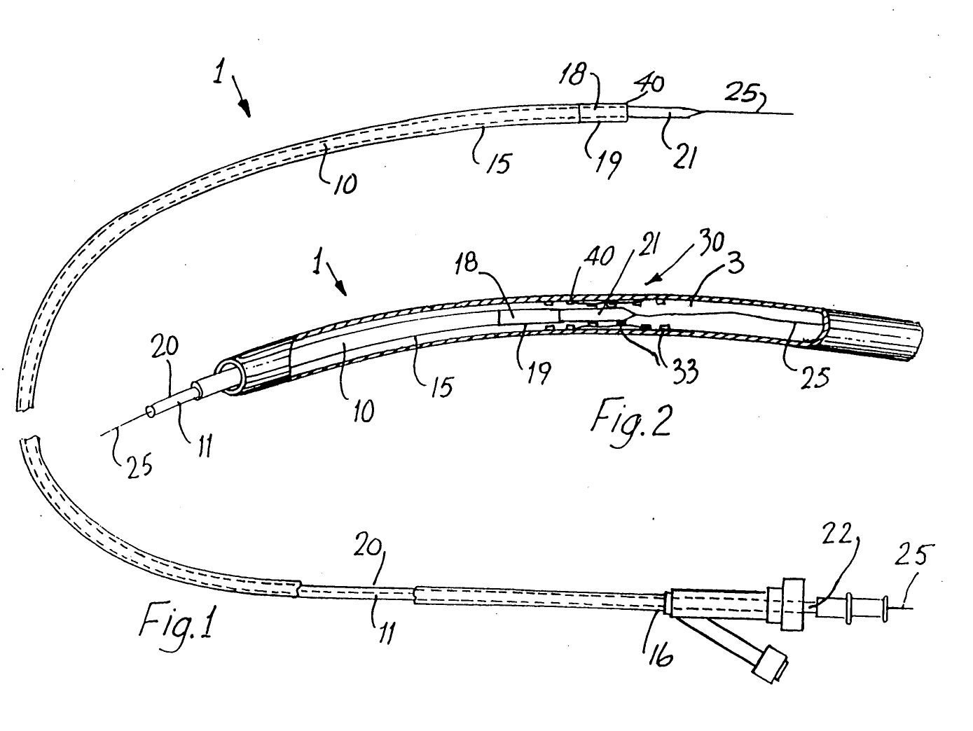

Referring to FIGS. 1 to 5 there is illustrated a retrieval device according to the invention and indicated generally by the reference numeral 1. The retrieval device 1 is used for retrieval of a medical device such as an embolic filter 2 from a body lumen such as a vasculature 3. The filter 2 is located distally of a lesion or another treatment device such as a stent. The embolic filter 2 is of the type described in our WO 99/23976A, the entire contents of which are incorporated herein by reference.

In use a guide catheter 42 (FIG. 4) extends through the body lumen 3 proximal to a stenosed region 30. The guide catheter 42 comprises an elongate tube having a proximal end and a distal end. The distal end has an open mouth 46. To retrieve the embolic filter 2 a retrieval device I is advanced through the guide catheter 42. The retrieval device 1 comprises a retrieval catheter 10 and a centring catheter 11.

The retrieval catheter 10 comprises an elongate flexible tube 15 having a proximal end 16 external of the lumen and fitted, for example with a Y-connector and a Tuohy Borst adapter. The retrieval catheter 10 extends through the guide catheter 42 and terminates beyond the distal end thereof. A distal end 18 of the retrieval catheter has an expansible tip 19 with an open mouth 40 to accommodate the embolic filter 2.

The centring catheter 11 comprises an elongate tube 20 which extends through the retrieval catheter tube 15 and terminates beyond the distal end 18 thereof in a soft, flexible tapered end 21. A proximal end 22 of the centring catheter 11 extends from the body lumen for external manipulation of the centring catheter 11. In this case the centring catheter tube 20 has a central bore for threading over a guidewire 25 to which the filter 2 may be mounted. The bore may extend only partially along the length of the centring catheter 11 to provide for rapid exchange. For ease of location, preferably distal end 21 of the centring catheter 11 is at least partially of, or coated with, a radiopaque material. The centring catheter 11 is of a suitable low friction material for smooth slidable travel through the retrieval catheter 10. A particularly suitable material for the centring catheter 11 would be a high density polyethylene/low density polyethylene (HDPE/LDPE) mix.

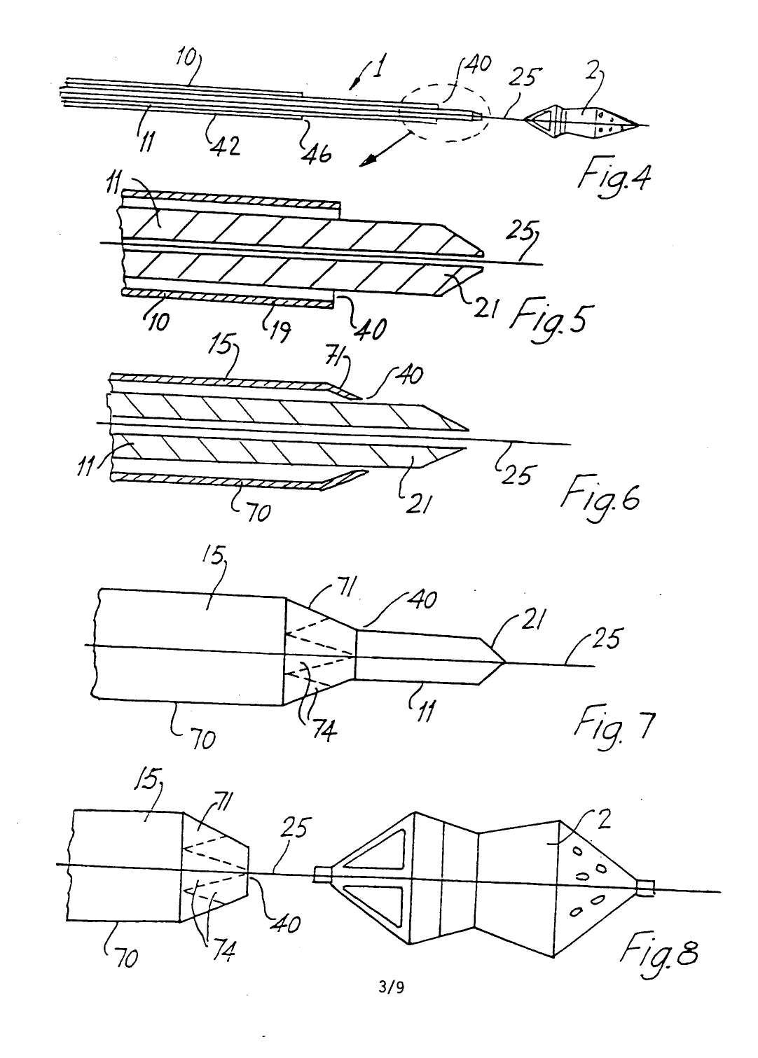

The tapered distal end 21 of the centring catheter 11, in use, projects from the open mouth 40 of the retrieval catheter 10 (FIG. 5). The soft, tapered distal end 21 allows the retrieval catheter 10 to be smoothly advanced through the vasculature 3 without becoming snagged on a partial blockage such as a region of stenosis, or on a deployed stent 33, or in stenosed regions 31, 32 adjacent to the stent 33. The taper may be straight as illustrated in FIG. 5, or may be curved in a concave manner.

To collapse the filter 2 after use and retrieve it from the body lumen 3, the retrieval catheter 10 is introduced over the guidewire 25 on which the filter 2 is mounted, in this case. With the aid of radiopaque markers, the filter 2 is urged into the retrieval catheter 10 by pulling the guidewire 25 proximally. The tip 19 of the retrieval catheter 10 is radially flexible/expansible to accommodate the embolic bulk that the filter 2 may have retained, but also has enough axial stiffness to assist the pull back of the filter 2 into the tip 19.

Alternatively the filter 2 may be retrieved into the tip 19 by advancing the retrieval catheter 10 further distally relative to the filter 2, thereby collapsing the filter 2.

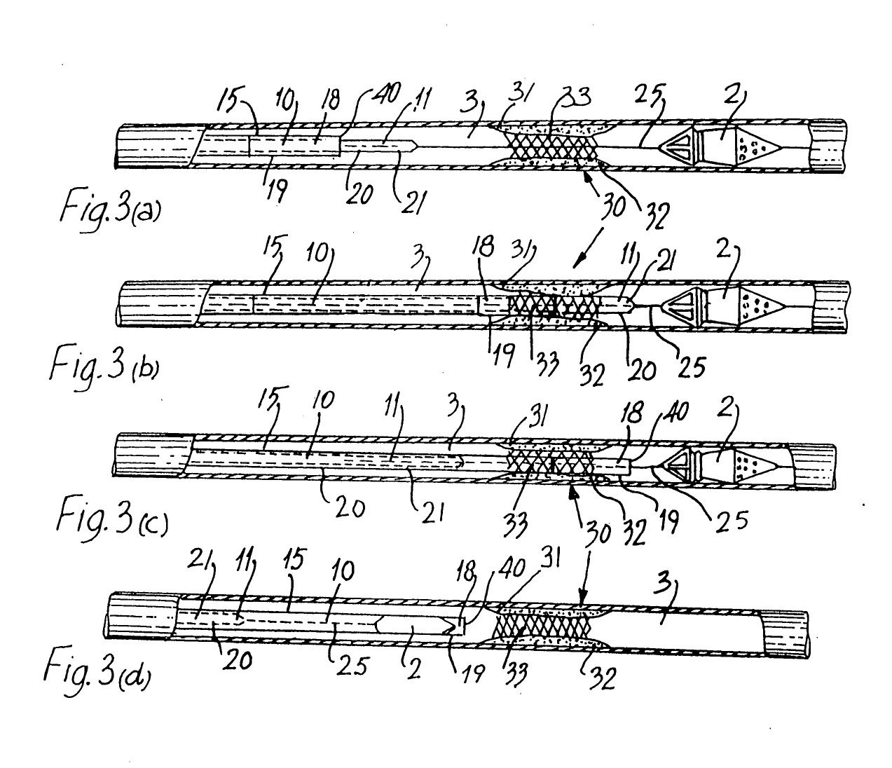

Referring in particular to FIGS. 3(a) to 3(d), in an advancement mode the tapered distal end 21 of the centring catheter 11 projects from the open distal end 18 of the retrieval catheter 10 over the guidewire 25 (FIG. 3(a)). The tapered distal end 21 of the centring catheter 11 guides the retrieval device 1 through the deployed stent 33 without snagging the retrieval catheter 10 on the stent 33 or the stenosed region 30 (FIG. 3(b)). When the retrieval catheter 10 has been advanced so that the distal end 21 of the centring catheter 11 is in a position adjacent the proximal end of the filter 2, the tapered end 21 of the centring catheter 11 is retracted proximally into the retrieval catheter 10, exposing the open mouth 40 of the retrieval catheter 10 (FIG. 3(c)). The filter 2 is then collapsed and retrieved into the expansible tip 19 of the retrieval catheter 10. The retrieval catheter 10 with the retrieved filter 2 in place is then withdrawn through the lumen 3 (FIG. 3(d)).

FIGS. 6 to 9 show another embodiment of the retrieval catheter, which is similar to the retrieval catheter 10 of FIGS. 1 to 5. In this case a retrieval catheter 70 comprises an elongate tube 15 with a tapered expandable tip 71 at the distal end of the tube 15. An open mount 40 is provided at the distal end of the tip 71 to receive the retrieved embolic filter 2 within the tip 71. The distal end of the tube 15 terminates in a plurality of splined fingers 74, over which the tip 71 is attached.

The tip 71 is of a flexible elastic polymeric material, for example polyurethane or a flexible grade of PEBA and the tube 15 is of a stiff material, for example a stiff grade of PEBAX. The tip 71 may be attached over the fingers 74 of the tube 15 by welding or by overmoulding.

FIG. 7 illustrates the advancement mode for the retrieval device, in which the distal end 21 of the centring catheter 11 projects from the open mouth 40 of the tip 71. The filter 2 is retrieved into the tip 71 by pulling the guidewire 25 proximally.

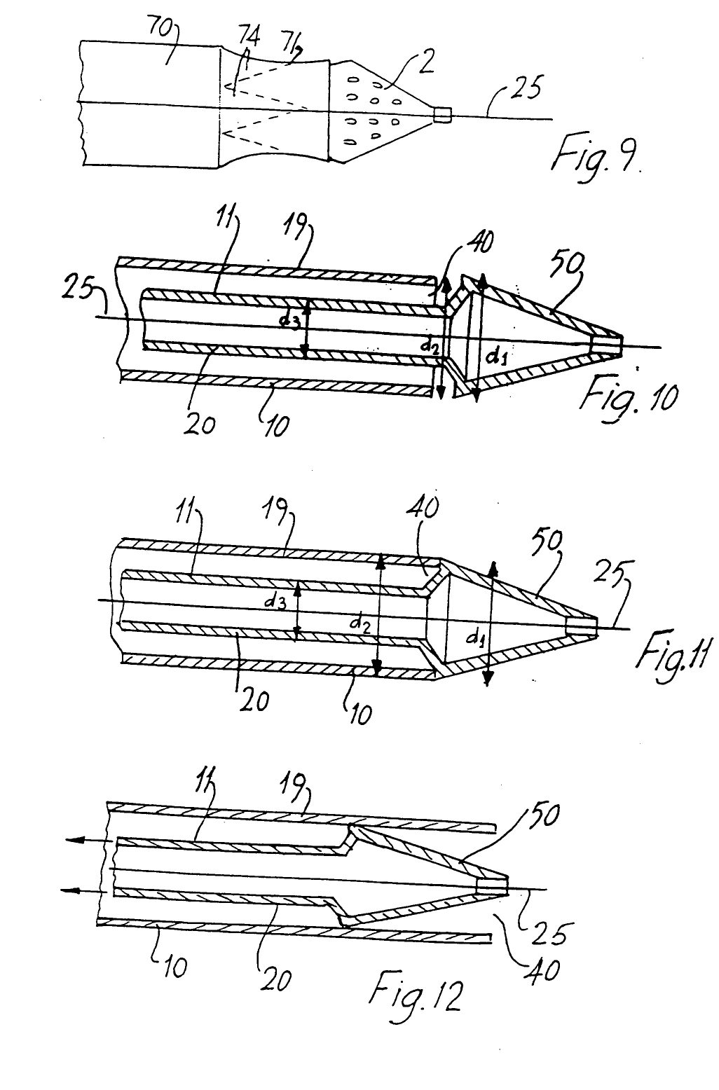

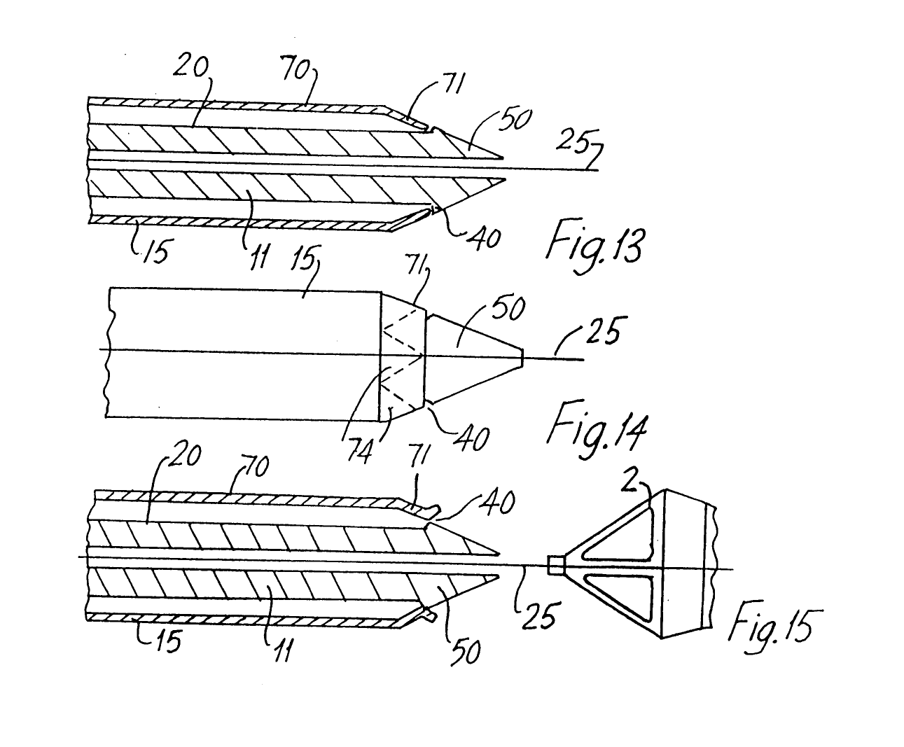

The tapered tip 71 expands outwardly as the filter 2 is retracted into the tip 71 to fully accommodate the retained embolic bulk within the filter 2, as illustrated in FIGS. 8 and 9.

The filter 2 may alternatively be retrieved into the tip 71 by advancing the retrieval catheter 70 distally relative to the filter 2.

The tip 71 is sufficiently expansible to facilitate retrieval of the filter 2, even with a large embolic load retained within the filter 2.

The tapered profile of the expansible tip 71 provides a smooth, step-free transition between the centring catheter 11 and the retrieval catheter 70. This prevents hang-up of the open mount 40 of the retrieval catheter 70 or causing further vessel trauma, as the retrieval device is advanced through the vasculature 3, in particular as the retrieval device crosses the stent 33 and stenosed regions 31, 32.

Referring now to FIGS. 10 to 12 in another embodiment of the invention, the distal end of the centring catheter tube 20 is in the shape of an arrowhead 50. The largest outer diameter d₁ of the distal end 50 of the centring catheter 11 is equal to the outer diameter d₂ of the retrieval catheter tip 19 at the open mouth 40. This ensures that there is a smooth, step free crossing profile between the centring catheter distal end 50 and the retrieval catheter tip 19 in the advancement mode of FIG. 11. Also the distal end 50 of the centring catheter tube 20 sealingly engages the retrieval catheter tip 19 in the advancement mode. This enables a centring catheter tube 20 with a diameter d₃ which is smaller than d₁ to be used, thus providing more lumen space between the centring catheter tube 20 and the retrieval catheter 10 to facilitate flushing and/or aspiration.

The distal end 50 of the centring catheter tube 20 is of a soft, flexible material, and is hollow. The distal end 50 may be deformed into a smaller diameter by pulling the centring catheter 11 proximally into the retrieval catheter 10 as illustrated in FIG. 12. This enables the smooth pull back of the centring catheter 11 into the retrieval catheter 10 prior to retrieval of the filter 2 into the tip 19. Pull back of the centring catheter 11 is further assisted by the axial stiffness of the tip 19 of the retrieval catheter 10.

A further embodiment of the invention is illustrated in FIGS. 13 to 17, in which the tapered distal tip 71 of the retrieval catheter 70 tapers towards the arrowhead-shaped distal end 50 of the centring catheter 11. Thus, in the advancement mode the retrieval device has a smooth, step-free crossing profile, as illustrated in FIGS. 13 and 14.

The tip 71 is radially expansible to enable retraction of the centring catheter tip 50 through the open mouth 40 proximally into the retrieval catheter 70 before retrieval of the filter 2 (FIG. 15), and then to enable retraction of the filter 2 together with the retained embolic load by pulling the guidewire 25 proximally (FIGS. 16 and 17).

The filter 2 may alternatively be retrieved into the tip 71 by advancing the retrieval catheter 70 distally relative to the filter 2 and/or centring catheter 11.

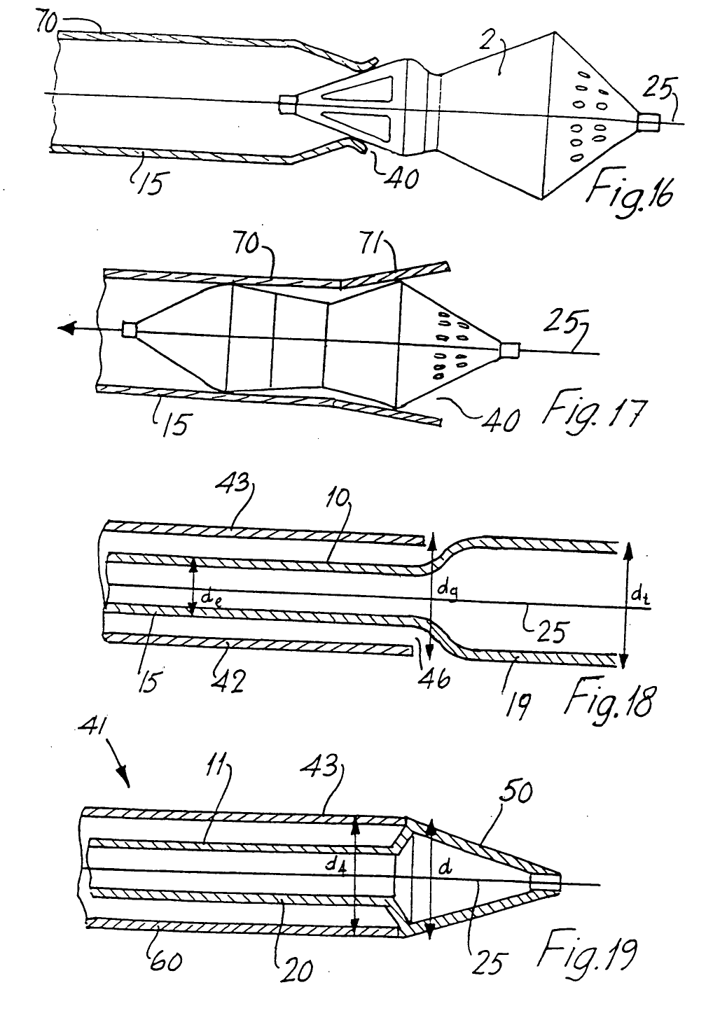

Referring now to FIG. 18 in another embodiment of the invention the diameter of the retrieval catheter 10 varies along its longitudinal length. In this case the diameter dₜ of the tip 19 of the retrieval catheter 10 is greater than the diameter dₑ of the remaining length of the retrieval catheter tube 15. The diameter dₜ of the tip 19 of the retrieval catheter 10 is equal to or greater than the diameter d_{g} of the guide catheter 42. This allows a retrieval catheter tube 15 of smaller diameter to be used, thus providing more lumen space between the retrieval catheter tube 15 and the guide catheter 42. This extra lumen space enables the injection of contrast media and the like through the guide catheter 42.

Referring to FIG. 19 there is illustrated another retrieval device 41 according to the invention, which is similar to the retrieval device 1 and like parts are assigned the same reference numerals. In this case the retrieval catheter 41 comprises a guide catheter 60 and a centring catheter 11, and a separate retrieval catheter is not required. The guide catheter 60 is of sufficiently small diameter to allow it to be advanced to the site of the stenosis 30, and the distal end 50 of the centring catheter 11 in the shape of an arrowhead is sized to match the outer diameter of the guide catheter 60. The largest outer diameter d₅ of the distal end 50 is equal to the outer diameter d₄ of the guide catheter 60. This ensures a smooth, step-free transition between the centring catheter 11 and the guide catheter 60.

The invention provides a retrieval system, which is simple to operate and yet provides snag-free manipulation of a retrieval catheter to a retrieval site. A smooth, step-free crossing profile is provided with a tapered transition to enable the retrieval catheter to pass through the diseased vessel and cross the stent without gouging the vessel, without catching on or dislodging the stent and without scraping or dislodging embolic material from the wall of the vessel or stent.

The centring catheter 11 also provides support to the retrieval catheter assembly, greatly enhancing the integrity and kink resistance of the system, so that an ultra low profile, thin wall retrieval catheter 10 can be used without compromising the integrity of the system. This provides a very large lumen within the tip 19 of the retrieval catheter 10 to facilitate the retrieval of large filters with large volumes of captured embolic material. In one embodiment of the invention the centring catheter has an inner diameter of 0.76 mm and an outer diameter of 1.63 mm, and the retrieval catheter has an inner diameter of 1.78 mm and an outer diameter of 2.08 mm. These dimensions are given as examples only and are by no means essential to the invention. It will be appreciate that other diameters may also be used to obtain a retrieval system with structural integrity and a large retrieval lumen.

The centring catheter 11 also maintains the guidewire 25 positioned towards the centre of the lumen 3 away from the walls of the lumen 3. This minimises the contact between the guidewire 25 and the walls of the lumen 3 to avoid further vessel trauma or release of embolic material from the vessel wall.

The invention has been described in relation to a carotid angioplasty procedure with an intravascular filter being placed distally to capture any emboli being released during the procedure. However, it may be applied to other medical procedures such as angioplasty and stenting of surgically implanted Saphenous Vein grafts that have stenosed, or primary treatment of renal artery stenoses. Indeed, the invention is applicable to the retrieval of embolic protection devices to protect patients during any vascular intervention. The invention may also be used for the retrieval of other medical devices, especially where there is a requirement for safe retrieval through an implanted stent.

Preferably the retrieval device 1 includes means to facilitate flushing and/or aspiration. In one embodiment of the invention, the centring catheter 11 may be withdrawn fully from the retrieval catheter 10, to facilitate flushing and/or aspiration.

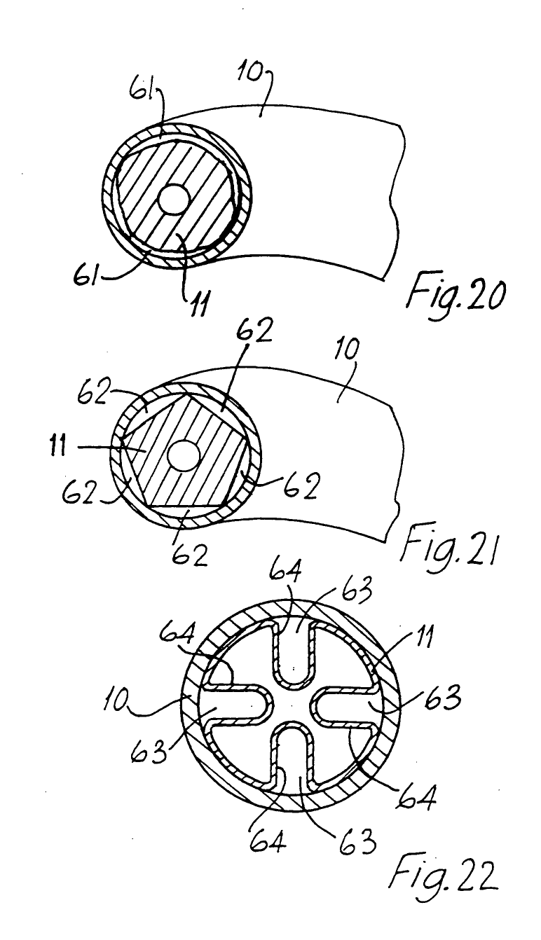

Referring to FIGS. 20 to 22 there are illustrated alternative centring catheters 11 with non-circular cross sections to facilitate flushing and/or aspiration without requiring the centring catheter 11 to be removed from the retrieval catheter 10. The non-circular cross section also reduces the area of contact between the centring catheter 11 and the retrieval catheter 10, thereby reducing the frictional force acting between the outer surface of the centring catheter tube 20 and the inner surface of the retrieval catheter tube 15.

Referring in particular to FIG. 20 in this case the centring catheter 11 is of a generally oval cross section. This creates defined passageways 61 between the retrieval catheter 10 and the centring catheter 11.

In the arrangement illustrated in FIG. 21 there are five contact points between the centring catheter 11 and the retrieval catheter 10, and again defined passageways 62 are created.

The fluted arrangement of FIG. 22 is preferred because defined passageways 63 are created between the flutes 64 while ensuring a large lumen space between the centring catheter 11 and the retrieval catheter 10.

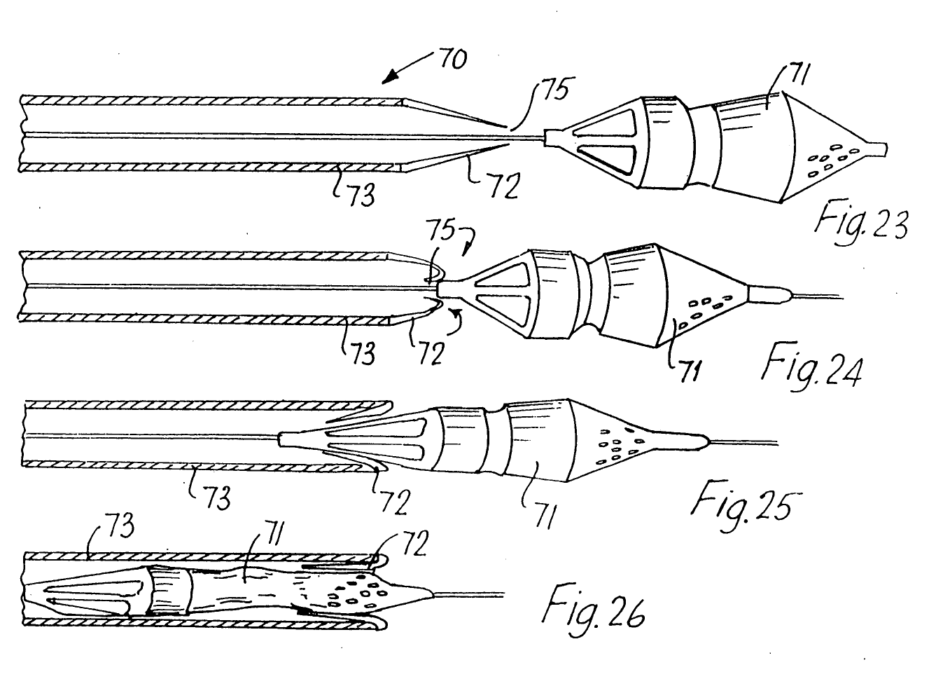

Referring to FIGS. 23 to 26 there is illustrated another retrieval device 70 for retrieving a medical device such as a distal embolic filter 71 located distally of a lesion or a treatment device. The retrieval device 70 has a centring means in the form of a tapered distal extension 72 of a main body of a retrieval catheter 73. The tapered distal extension 72 has an open mouth 75 through which the filter 71 is retrieved.

In this case at least portion of the distal extension 72 is movable inwardly in the direction of the arrows in FIG. 24 between an introduction configuration as illustrated in FIG. 23 in which the retrieval device crosses a lesion or another treatment device such as a stent and is advanced to the filter 71 . As the filter 71 is pulled back and collapses into the retrieval catheter 23 the distal extension 72 is forced inwardly into the retrieval catheter into the final retrieval configuration illustrated in FIG. 26 far withdrawn from the body lumen.

The distal extension 72 may be of the same material as that of the main body 73 of the retrieval catheter but with substantially reduced wall thickness. Alternatively or additionally the extension may be of a resilient elastomeric material such as a soft polyurethane, PEBAX or PTFE.

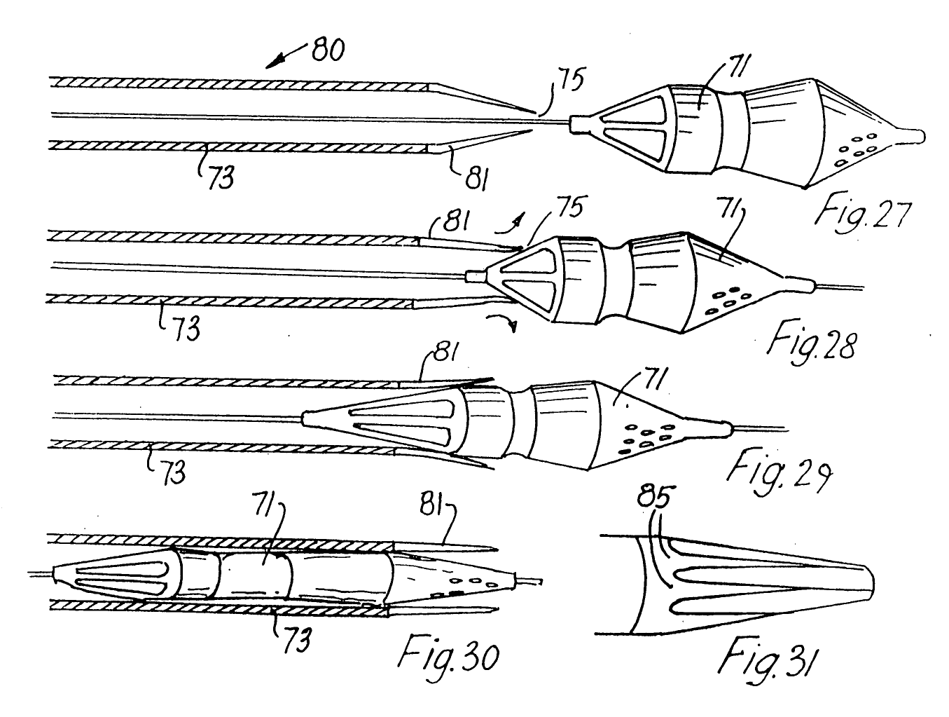

Referring to FIGS. 27 to 31 there is illustrated another retrieval device 80 similar to the device of FIGS. 23 to 26 and like parts are assigned the same reference numerals. In this case the distal extension 81 is movable radially outwardly between the introduction and retrieval configurations. The tapered distal extension 81 may be similar to the arrangement of FIGS. 6 to 9. Ideally the extension 81 has a number of tapered reinforcing legs 85 covered in a softer elastic material forming a conical but expansible pod for the reception of the filter 70. The rigid reinforcing legs 85 ensure the tip of that the distal extension is not inverted and assist in centring the guidewire.

The invention is not limited to the embodiments hereinbefore described, which may be varied in detail.

1-48. (canceled)

49. A filter retrieval device, comprising:
a catheter including an elongate tube having a proximal end and a distal end and a housing disposed at the distal end, the housing having a distal end;
a guidewire extending at least in part through the catheter, the guidewire having a proximal end and a distal end;
a filter connected to the guidewire proximate the distal end of the guidewire, the filter is moveable from a position outside the housing to a position at least in part inside the housing; and
a centering catheter disposed within the housing and extending distally therefrom when the filter is in the position outside the housing.

50. The filter retrieval device in accordance with claim 49, wherein the centering catheter tapers distally.

51. The filter retrieval device in accordance with claim 49, wherein the centering catheter is releasably engaged to the distal end of the housing.

52. A filter retrieval device, comprising:
a catheter including an elongate tube having a proximal end and a distal end; a housing disposed at the distal end of the elongate tube;
a guidewire extending at least in part through the catheter, the guidewire having a proximal end and a distal end;
a filter disposed at the distal end of the guidewire; and
a centering catheter disposed within the housing, the centering catheter having a distally disposed soft, flexible tip.

53. The filter retrieval device in accordance with claim 52, wherein the centering catheter tapers distally.

54. The filter retrieval device in accordance with claim 52, wherein the centering catheter is releasably engaged to the distal end of the housing.

55. The filter retrieval device in accordance with claim 52, wherein the centering catheter includes a balloon.

56. The filter retrieval device in accordance with claim 52, wherein the guidewire extends distally of the filter.

57. The filter retrieval device in accordance with claim 56, further comprising a spring tip at the distal end of the guidewire.

58. The filter retrieval device in accordance with claim 52, wherein the filter includes an expandable frame and a filter membrane.

59. The filter retrieval device in accordance with claim 58, wherein the frame includes a NiTi alloy.

60. The filter retrieval device in accordance with claim 58, wherein the filter membrane surrounds a portion of the guidewire.

61. The filter retrieval device in accordance with claim 58, wherein the filter membrane portion of the filter tapers distally.

62. The filter retrieval device in accordance with claim 58, wherein the filter membrane includes polyurethane.

63. The filter retrieval device in accordance with claim 58, wherein the filter membrane defines a plurality of apertures.

64. The filter retrieval device in accordance with claim 52, wherein the soft, flexible tip includes a proximal end and a distal end.

65. The filter retrieval device in accordance with claim 64, wherein the proximal end of the soft, flexible tip includes an engagement ledge.

66. The filter retrieval device in accordance with claim 64, wherein the distal end of the soft, flexible tip is tapered.

67. A filter retrieval device, comprising:
a catheter including an elongate tube having a proximal end and a distal end; a housing disposed at the distal end of the elongate tube;
a guidewire extending at least in part through the catheter, the guidewire having a proximal end and a distal end;
a filter disposed at the distal end of the guidewire, the filter includes an expandable frame and a filter membrane; and
a centering catheter disposed within the housing, the centering catheter having a distally disposed soft, flexible tip wherein the proximal end of the soft, flexible tip includes an engagement ledge and the distal end of the soft, flexible tip is tapered.

68. The filter retrieval device in accordance with claim 67, wherein the centering catheter tapers distally.

69. The filter retrieval device in accordance with claim 67, wherein the centering catheter is releasably engaged to the distal end of the housing.

70. The filter retrieval device in accordance with claim 67, wherein the centering catheter includes a balloon.

71. The filter retrieval device in accordance with claim 67, wherein the guidewire extends distally of the filter.

72. The filter retrieval device in accordance with claim 71, further comprising a spring tip at the distal end of the guidewire.

73. The filter retrieval device in accordance with claim 67, wherein the frame includes a NiTi alloy.

74. The filter retrieval device in accordance with claim 67, wherein the filter membrane surrounds a portion of the guidewire.

75. The filter retrieval device in accordance with claim 67, wherein the filter membrane portion of the filter tapers distally.

76. The filter retrieval device in accordance with claim 67, wherein the filter membrane includes polyurethane.

77. The filter retrieval device in accordance with claim 67, wherein the filter membrane defines a plurality of apertures.
